(19) United States
(12) Patent Application Publication
(10) Pub. No.: US 2013/0123859 A1
(43) Pub. Date: May 16, 2013

(54) CERVICAL SPINAL SYSTEM

(71) Applicants: Jeffrey GROSS, Coto de Caza, CA (US); Ryan SINGH, Loxahatchee, FL (US); J. Scott HAY, Parkland, FL (US)

(72) Inventors: Jeffrey GROSS, Coto de Caza, CA (US); Ryan SINGH, Loxahatchee, FL (US); J. Scott HAY, Parkland, FL (US)

(21) Appl. No.: 13/666,636

(22) Filed: Nov. 1, 2012

Related U.S. Application Data
(60) Provisional application No. 61/554,178, filed on Nov. 1, 2011.

(51) Int. Cl. A61B 17/80; A61B 17/86

(52) U.S. Cl. CPC A61B 17/80; A61B 17/861
USPC 606/305; 606/289

(57) ABSTRACT

An orthopedic fixation system, including a tissue anchor including a first instrument engagement feature and a second instrument engagement feature; a first tool engageable with the first instrument engagement feature, and a second tool engageable with the second instrument engagement feature, where the first and second tools are engageable simultaneously with the tissue anchor.

### CROSS-REFERENCE TO RELATED APPLICATION

This application is related to and claims priority to U.S. Provisional Patent Application Serial No. 61/554,178, filed Nov. 1, 2011, entitled US Provisional Patent for CERVICAL SPINAL SYSTEM, the entirety of which is incorporated herein by reference.

### STATEMENT REGARDING FEDERALLY SPONSORED RESEARCH OR DEVELOPMENT

n/a

### FIELD OF THE INVENTION

The present invention relates to a methods and systems for anterior spinal fixation or fusion of the cervical spine, and in particular to plate fixation systems for aligning and maintaining adjacent cervical vertebrae during spinal fusion of those vertebrae.

### BACKGROUND OF THE INVENTION

Current practices in orthopedic surgery employ plating systems for joining portions of a broken bone, or for fusion of portions of separate bones. Such systems are composed essentially of plates and screws for aligning and holding the bone portions in a desired position relative to one another. Plating systems have particular usefulness in the spine, and have general skeletal use on the flat bones, such as the scapula and the pelvis by way of example, and for use on tubular bones, such as the humerus, ulna, radius, femur, and tibia by way of example.

However, problems associated with such plating systems typically include hardware breakage, hardware loosening, inability to gain adequate fixation, and distraction pseudoarthrosis where the plate prevents bone portions from coming together over time, which impeded or altogether prevents healing. These occurrences cause additional problems to a patient, and can require further surgical procedures to repair the damage, remove the failed hardware, and/or to reattempt skeletal stabilization.

Based on a consideration of such drawbacks, there remains a need for an improved plating system providing sufficiently durable hardware to perform their intended function without mechanical failure and the ability to reliably and easily engage bone screws or anchors to the plate with minimal interference between instrumentation used to implant, position, or otherwise anchor the fixation components to the tissue.

### SUMMARY OF THE INVENTION

The present disclosure advantageously provides an orthopedic fixation system, including: a tissue anchor including a first instrument engagement feature and a second instrument engagement feature; a first tool engageable with the first instrument engagement feature, and a second tool engageable with the second instrument engagement feature, where the first and second tools are engageable simultaneously with the tissue anchor. The first tool may be positionable coaxially within the second tool. The tissue anchor may be an orthopedic screw defining a head and a threaded body. The first and second instrument engagement features are on the head of the tissue anchor. The second instrument engagement feature may circumscribe at least a portion of the first instrument engagement feature. The first instrument engagement feature may include a threaded cavity. The system may include a locking element releasably engageable with the first instrument engagement feature, where the locking element may include a set screw threadably engageable with the first instrument engagement feature. The system may include a bone plate defining a plurality of openings therethrough, where the bone plate is securable to the tissue anchor with the locking element.

A surgical kit is also provided, including a fixation plate defining a plurality of openings therethrough; a bone anchor defining a first instrument engagement feature and a second instrument engagement feature; a first tool passable through one of the plurality of openings in the fixation plate and engageable with the first instrument engagement feature, and a second tool passable through one of the plurality of openings in the fixation plate and engageable with the second instrument engagement feature, wherein the first and second tools are engageable with the respective instrument engagement features simultaneously. The second tool may be slidably positionable over the first tool. The second instrument engagement feature may circumscribe at least a portion of the first instrument engagement feature. The first instrument engagement feature may include a threaded cavity. The surgical kit may further include a locking element engageable with the first instrument engagement feature to secure the fixation plate to the bone anchor.

A medical method is provided, including coupling a first instrument to a tissue anchor; inserting the tissue anchor into a portion of tissue with the first instrument; positioning a fixation plate adjacent the tissue anchor; positioning a second instrument coaxially with the first instrument; engaging the tissue anchor with the second instrument while the first instrument remains coupled to the tissue anchor; decoupling the first instrument from the tissue anchor without substantially moving the tissue anchor; disengaging the second instrument from the tissue anchor; and securing the fixation plate to the tissue anchor. Securing the fixation plate may include affixing a set screw to the tissue anchor. The portion of tissue may include a spinal segment. The tissue anchor may include a first instrument engagement feature and a second instrument engagement feature circumscribing at least a portion of the first instrument engagement feature. The fixation plate may define a plurality of openings therethrough, and wherein positioning the fixation plate includes passing the first instrument through one of the plurality of openings. The tissue anchor may be an orthopedic screw.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is an illustration of an example of an anchor and associated instrumentation of a spinal stabilization system constructed in accordance with the principles of the present disclosure;

FIG. 1A is an illustration of another example of an anchor of a spinal stabilization system constructed in accordance with the principles of the present disclosure;

FIG. 2 is an illustration of an example of an anchor, associated instrumentation, and plate of a spinal stabilization system constructed in accordance with the principles of the present disclosure;

FIG. 3 is an illustration of an example of an anchor, plate, and associated instrumentation of a spinal stabilization system constructed in accordance with the principles of the present disclosure;

FIG. 4 is another illustration of an example of an anchor, plate, and associated instrumentation of a spinal stabilization system constructed in accordance with the principles of the present disclosure;

FIG. 5 is yet another illustration of an example of an anchor, plate, and associated instrumentation of a spinal stabilization system constructed in accordance with the principles of the present disclosure;

FIG. 6 is still another illustration of an example of an anchor, plate, and associated instrumentation of a spinal stabilization system constructed in accordance with the principles of the present disclosure;

FIG. 7 is an illustration of an example of an anchor and plate of a spinal stabilization system constructed in accordance with the principles of the present disclosure; an

FIG. 8 is an illustration of an example of an anchor, plate, and locking element of a spinal stabilization system constructed in accordance with the principles of the present disclosure.

### DETAILED DESCRIPTION OF THE INVENTION

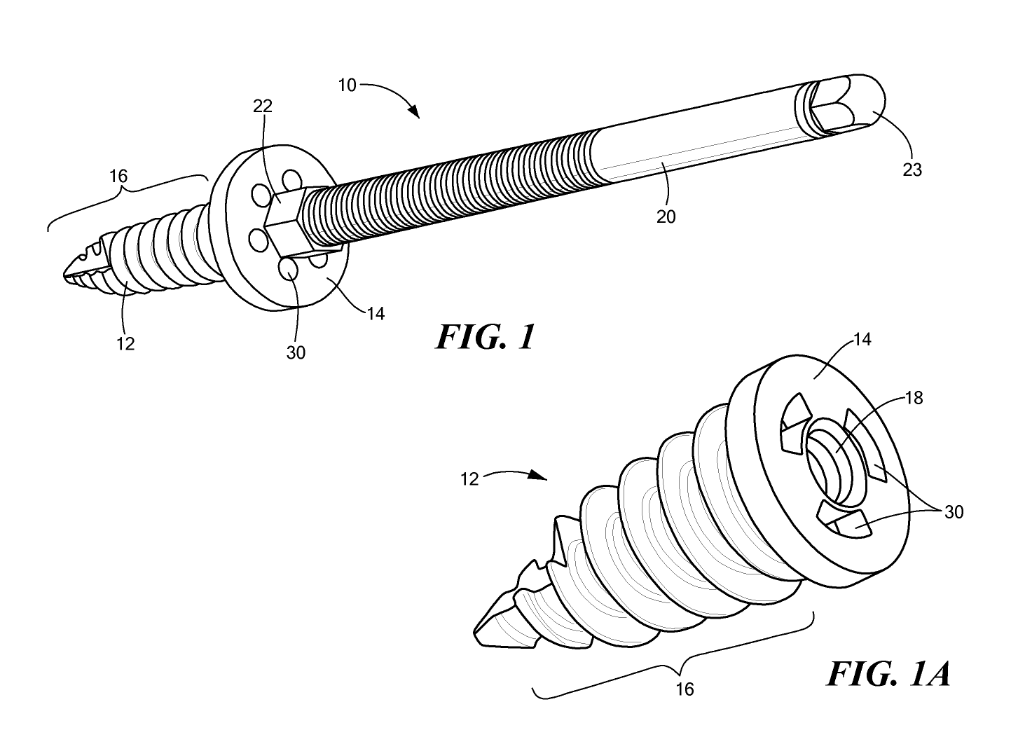

The present disclosure provides an orthopedic fixation or spinal stabilization system and methods of use thereof for accurately fusing or fixating selected potions of a spinal segment. Referring now to the drawing figures in which like reference designations refer to like elements, an embodiment of an orthopedic fixation or spinal stabilization system is shown in FIGS. 1-8 and generally designated as ‘10.’ Of note, the components of the fixation system 10 are illustrated and described herein with respect to one or more vertebrae or other segments of a spinal column, which are omitted from the figures for simplicity.

Now referring to FIGS. 1 and 1A, the fixation system 10 generally includes a tissue or bone anchor 12 engageable with a portion of a spinal column or vertebrae. The bone anchor 12 may generally define a head 14 opposite a threaded body 16 which is securely positionable within a designated tissue site. The head 14 of the bone anchor 12 may define a first tool or instrument engagement feature 18 that is releasably engageable with a first tool or driver 20 to facilitate the rotatable engagement of the bone anchor 12 into the tissue. The first engagement feature 18 may include, for example, a threaded hole or cavity that matably couples to a threaded end of the first tool 20.

The first tool 20 may generally define an elongated body having a proximal portion graspable or otherwise accessible to a surgeon or user during a medical procedure. The elongated body of the first tool 20 may further define a distal end or portion engageable with the first engagement feature 18 of the anchor 12. The first tool 20 may define a shoulder 22 that limits the length of engagement between the head 14 of the bone anchor 12 and the first tool 20, which also provides a degree of tactile feedback to a user or surgeon indicating when the bone anchor 12 and the first tool 20 are securely and fully engaged. The elongated shaft or body of the first tool 20 may extend away from the shoulder and the bone anchor 12, where the proximal portion 23 of the first tool 20 opposite the shoulder is sized or shaped to couple to an actuator, such as a handle, drill, or the like, as discussed in more detail below.

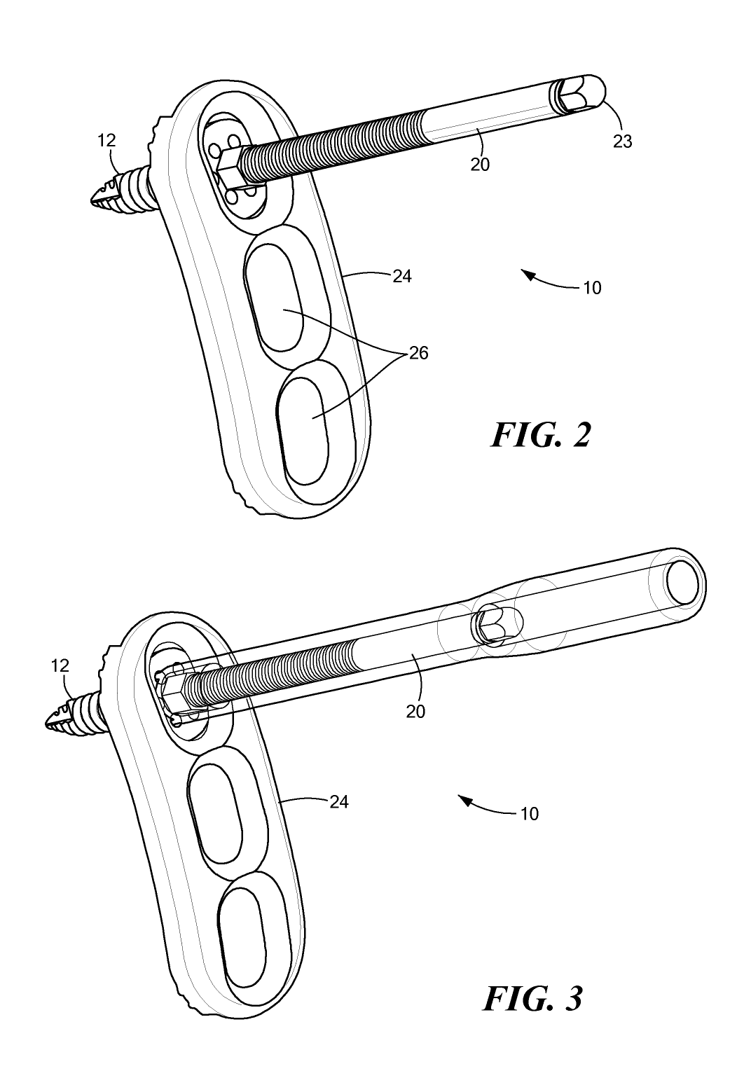

Now referring to FIG. 2, the first tool 20 may be used to securely position the bone anchor 12 into a desired orientation within a tissue region. Once in a desired location, a fusion or fixation plate 24 may be positioned over the first tool 20 and adjacent to the bone anchor 12. One or more openings 26 in the fixation plate 24 may at least partially circumscribe the head 14 of the bone anchor 12, thereby reducing any interference between the first tool 20 and the bone anchor 12. The fixation plate 24 may be sized and/or shaped to span one or more vertebral bodies, with one or more additional bone anchors (not shown) being used to anchor 12 remaining portions of the fixation plate 24 to the spinal segment or other region of the targeted tissue in the selected surgical site. Moreover, the fixation plate may define ridges or “teeth” on an underside thereof to aid in engaging or securing the plate to the targeted tissue.

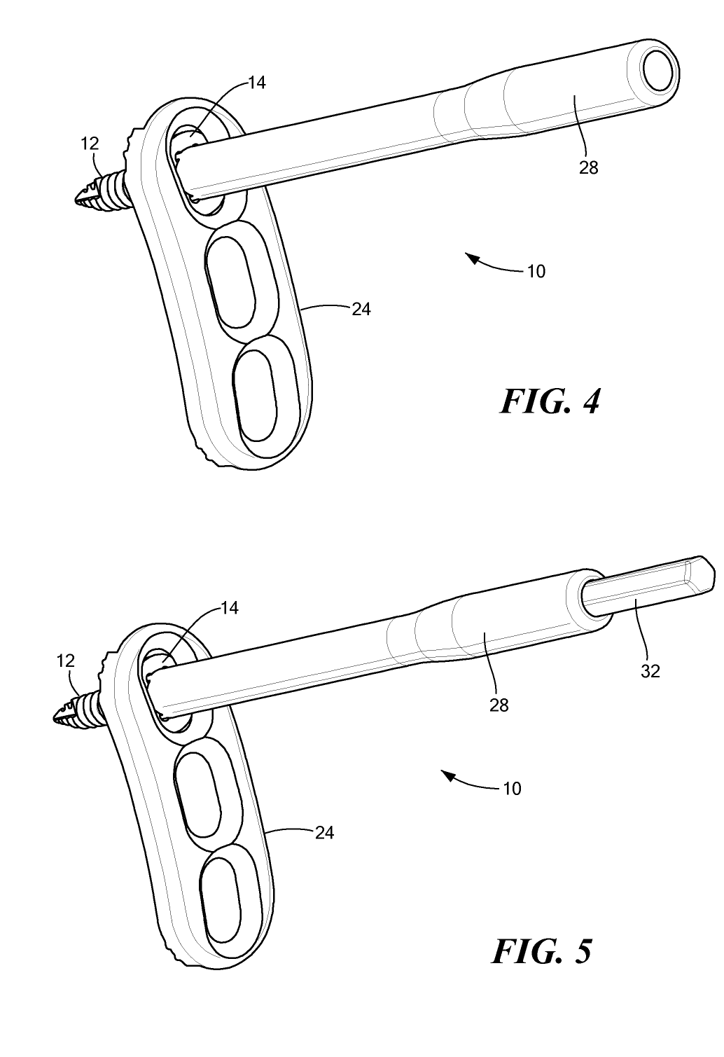

Turning to FIGS. 3-4, once the fixation plate 24 is in a desired position, a second tool or driver 28 may be coaxially disposed over the first tool 20 and engaged to the head 14 of the bone anchor 12. That is, the second tool 28 may be substantially hollow and/or define a passage therethrough that allows the second tool 28 to be positioned around the first tool 20. The second tool 28 may be releasably coupled to the head 14 of the bone anchor 12 through a second tool or instrument engagement feature 30 on the head 14 that is separate and distinct from the first engagement feature 18. The separate and independent nature of the first and second engagement features 18, 30 allows both the first and second drivers 20, 28 to couple to the head 14 of the bone anchor 12 simultaneously without limiting or interfering with their independent operation. For example, the second engagement feature 30 may include a plurality of depressions or cavities that circumscribe the first engagement feature 18, while the second tool 28 may define a complementary plurality of protrusions that are engageable with or otherwise positionable within the plurality of depressions or cavities. The engagement of the second tool 28 to the head 14 of the bone anchor 12 substantially joins any rotational movement of the two, i.e., once engaged, the bone anchor 12 will not rotate without also rotating the second tool 28.

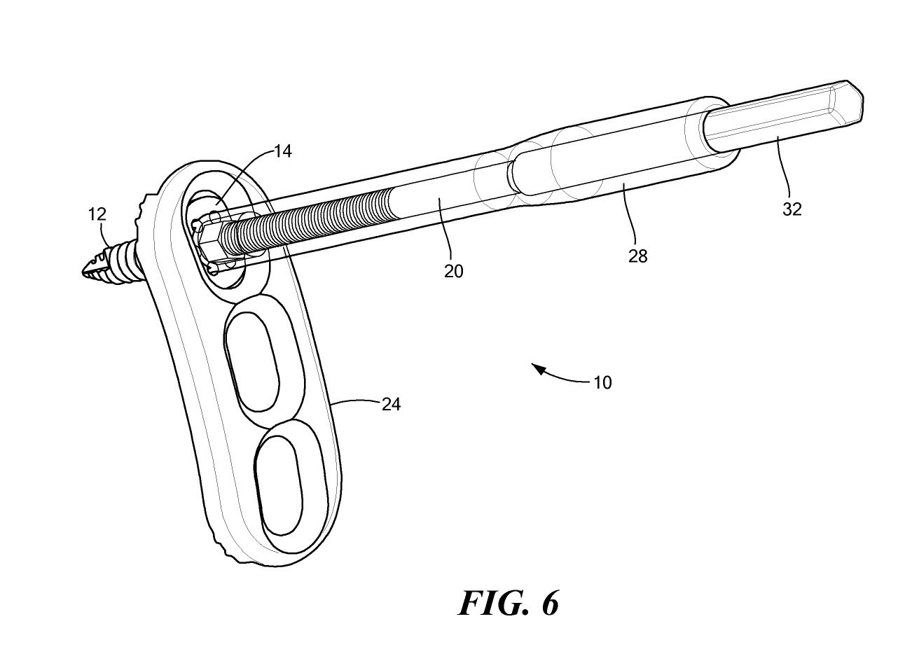

Now referring to FIGS. 5-6, upon securing the second tool 28 to the head 14 of the bone anchor 12, an actuator 32 may be passed through the passage of the second tool 28 to engage the first tool 20. The actuator 32 may include, for example, a handle, drill, drill bit, or other engaging structure to facilitate rotation or torqueing of the first tool 20. Once engaged, the actuator 32 may be rotated to release the engagement between the first tool 20 and the bone anchor 12, while the second tool 28 may be used to secure or otherwise hold the bone anchor 12 in place while the first tool 20 is decoupled. Securing the bone anchor 12 with the second tool 28 allows removal of the first tool 20 without loosening or backing out the bone anchor 12 and the positioning of the fixation plate 24.

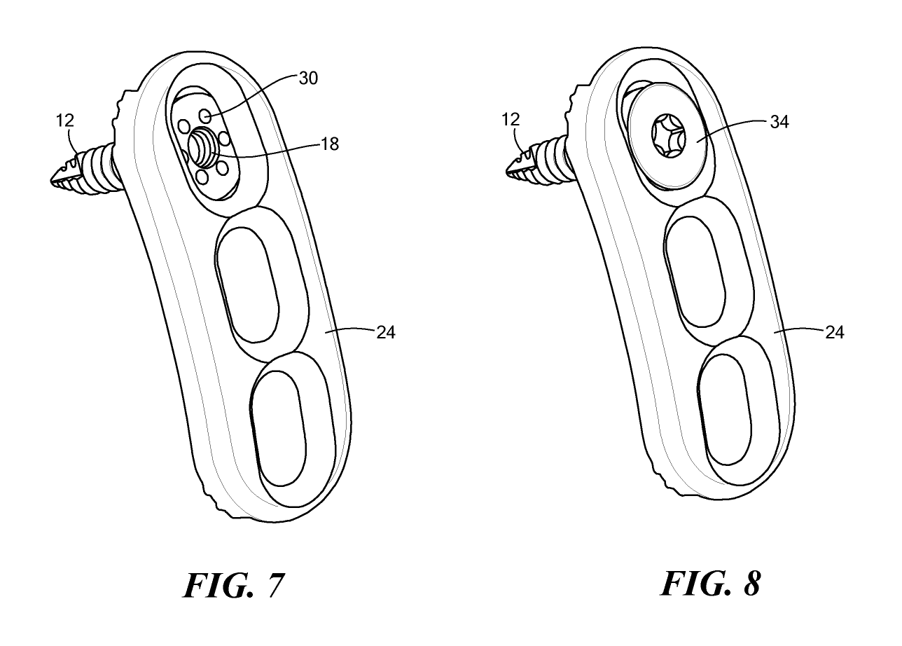

Turning to FIGS. 7-8, once the first tool 20 is removed, the second tool 28 may also be de-coupled from the bone anchor 12. As the second tool 28 was releasably coupled to the head 14 of the bone anchor 12 through a longitudinal direction (e.g., as opposed to rotatably threadably engaging the bone anchor 12), releasing the second tool 28 from the head 14 of the bone anchor 12 can be done through the application of a modest axial or longitudinal force, which is unlikely to disturb the engagement between the bone anchor 12 and the tissue.

Upon removal of the first and second drivers, a locking element 34 may be secured to the bone anchor 12 (by engaging the first engagement feature 18, for example), thereby also securing the fixation plate 24 to the bone anchor 12. The fixation plate 24 may be held in place by one or more plate fixation tools (not shown) during the securement of the locking element 34. Moreover, the securement of the fixation plate 24 and the locking element 34 may provide for a selected range of movement between one or more attached spinal regions, e.g., the fixation plate may be slidable about the locking element 34 and/or the anchor 12 upon implantation. Accordingly, the system may be used selectively to either fixate and/or dynamically couple two or more vertebrae with a desired range of motion, which may be defined at least in part by the dimensions of the openings, the locking element, and/or a clearance or compressive force exerted onto the fixation plate by the anchor 12 and/or the locking element 34.

It will be appreciated by persons skilled in the art that the present invention is not limited to what has been particularly shown and described herein above. In addition, unless mention was made above to the contrary, it should be noted that all of the accompanying drawings are not to scale. Of note, the system components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein. Moreover, while certain embodiments or figures described herein may illustrate features not expressly indicated on other figures or embodiments, it is understood that the features and components of the system and devices disclosed herein are not necessarily exclusive of each other and may be included in a variety of different combinations or configurations without departing from the scope and spirit of the invention. A variety of modifications and variations are possible in light of the above teachings without departing from the scope and spirit of the invention, which is limited only by the following claims.

What is claimed is:

1. An orthopedic fixation system, comprising:
a tissue anchor including a first instrument engagement feature and a second instrument engagement feature;
a first tool engageable with the first instrument engagement feature, and a second tool engageable with the second instrument engagement feature, wherein the first and second tools are engageable simultaneously with the tissue anchor.

2. The system of claim 1, wherein the first tool is positionable coaxially within the second tool.

3. The system of claim 1, wherein the tissue anchor is an orthopedic screw defining a head and a threaded body.

4. The system of claim 3, wherein the first and second instrument engagement features are on the head of the tissue anchor.

5. The system of claim 1, wherein the second instrument engagement feature circumscribes at least a portion of the first instrument engagement feature.

6. The system of claim 1, wherein the first instrument engagement feature includes a threaded cavity.

7. The system of claim 1, further comprising a locking element releasably engageable with the first instrument engagement feature.

8. The system of claim 7, wherein the locking element is a set screw threadably engageable with the first instrument engagement feature.

9. The system of claim 7, further comprising a bone plate defining a plurality of openings therethrough, wherein the bone plate is securable to the tissue anchor with the locking element.

10. A surgical kit, comprising:
a fixation plate defining a plurality of openings therethrough;
a bone anchor defining a first instrument engagement feature and a second instrument engagement feature;
a first tool passable through one of the plurality of openings in the fixation plate and engageable with the first instrument engagement feature, and a second tool passable through one of the plurality of openings in the fixation plate and engageable with the second instrument engagement feature, wherein the first and second tools are engageable with the respective instrument engagement features simultaneously.

11. The surgical kit of claim 10, wherein the second tool is slidably positionable over the first tool.

12. The surgical kit of claim 10, wherein the second instrument engagement feature circumscribes at least a portion of the first instrument engagement feature.

13. The surgical kit of claim 12, wherein the first instrument engagement feature includes a threaded cavity.

14. The surgical kit of claim 10, further comprising a locking element engageable with the first instrument engagement feature to secure the fixation plate to the bone anchor.

15. A medical method, comprising:
coupling a first instrument to a tissue anchor;
inserting the tissue anchor into a portion of tissue with the first instrument;
positioning a fixation plate adjacent the tissue anchor;
positioning a second instrument coaxially with the first instrument;
engaging the tissue anchor with the second instrument while the first instrument remains coupled to the tissue anchor;
decoupling the first instrument from the tissue anchor without substantially moving the tissue anchor;
disengaging the second instrument from the tissue anchor; and
securing the fixation plate to the tissue anchor.

16. The method of claim 15, wherein securing the fixation plate includes affixing a set screw to the tissue anchor.

17. The method of claim 15, wherein the portion of tissue includes a spinal segment.

18. The method of claim 15, wherein the tissue anchor includes a first instrument engagement feature and a second instrument engagement feature circumscribing at least a portion of the first instrument engagement feature.

19. The method of claim 15, wherein the fixation plate defines a plurality of openings therethrough, and wherein positioning the fixation plate includes passing the first instrument through one of the plurality of openings.

20. The method of claim 15, wherein the tissue anchor is an orthopedic screw.
